(19) United States
(12) Patent Application Publication
(10) Pub. No.: US 2016/0184614 A1
(43) Pub. Date: Jun. 30, 2016

(54) SYSTEMS AND METHODS FOR TREATING ABSCESSES AND INFECTED FLUID COLLECTIONS

(71) Applicant: UNIVERSITY OF WASHINGTON THROUGH ITS CENTER FOR COMMERCIALIZATION, Seattle, WA (US)

(72) Inventors: Thomas J. Matula, Seattle, WA (US); Keith Chan, Seattle, WA (US)

(21) Appl. No.: 14/907,443

(22) PCT Filed: Aug. 27, 2014
(86) PCT No.: PCT/US14/53001

§ 371 Date: Jan. 25, 2016

Related U.S. Application Data
(60) Provisional application No. 61/870,543, filed on Aug. 27, 2013.
(60) Provisional application No. 62/000,787, filed on May 20, 2014.
(60) Provisional application No. 62/042,682, filed on Aug. 27, 2014.

(51) Int. Cl. A61N 7/00; A61B 8/08; A61B 8/00; A61B 17/32

(52) U.S. Cl. CPC A61N 7/00; A61B 17/320068; A61B 8/085; A61B 8/4494; A61B 8/461; A61N 2007/0052; A61B 2090/374

(57) ABSTRACT

The present disclosure provides systems and methods for treating (e.g., reducing and/or eliminating) abscesses by applying acoustic energy, for example high intensity focused ultrasound (“HIFU”).

### CROSS-REFERENCE TO RELATED APPLICATIONS

This application claims the benefit of U.S. Provisional Patent Application No. 61/870,543, filed Aug. 27, 2013, U.S. Provisional Patent Application No. 62/000,787, filed May 20, 2014, and U.S. Provisional Patent Application No. 62/042,682, filed Aug. 27, 2014, each of which is incorporated herein by reference in its entirety.

### TECHNICAL FIELD

The present disclosure relates to systems and methods for treating (e.g., reducing and/or eliminating) abscesses and infected fluid collections by applying acoustic energy, for example high intensity focused ultrasound.

### BACKGROUND

Bacterial infection remains a global and ubiquitous healthcare problem, and can present within any body tissue or organ. Localized intra-abdominal or pelvic infections commonly occur due to the presence of foreign materials, or the entry of pathogens from adjacent the gastrointestinal or genitourinary tract into a previously-sterile bodily cavity, such as after surgery, trauma, inflammation, or external infections. Some infections may self-resolve, but many do not respond to antibiotic therapy alone. As these infected fluid collections (or “phlegmons”) evolve, they can develop into abscesses that require surgical or catheter-based drainage. Such treatment necessitates the use of an operating suite and advanced imaging and, in some instances, may require prolonged hospitalization.

### BRIEF DESCRIPTION OF THE DRAWINGS

Many aspects of the present technology can be better understood with reference to the following drawings. The relative dimensions in the drawings may be to scale with respect to some embodiments. With respect to other embodiments, the drawings may not be to scale. For ease of reference, throughout this disclosure identical reference numbers may be used to identify identical or at least generally similar or analogous components or features.

FIG. 1 is a partially schematic view of an acoustic abscess ablation system configured to administer therapeutic acoustic energy to an abscess of a patient or subject in accordance with an embodiment of the present technology

FIG. 2 is a partially schematic view of a method of applying HIFU energy to an abscess of a patient or subject using the system of FIG. 1.

FIG. 3 is a plot of relative bacterial activity after treatment with systems and methods configured according to embodiments of the present technology compared to untreated control, hot water bath treatment, and positive control.

FIG. 4A shows differential immunofluorescent stained sample of human pus before treatment. FIG. 4B shows differential immunofluorescent stained sample of the human pus after in vitro treatment with HIFU for 3 minutes.

FIG. 5 shows a fluoroscopic image of an induced abscess in a rabbit model.

FIGS. 6A and 6B show ultrasound images of a heterogeneous, partly hypoechoic mass induced in a rabbit model.

FIG. 7 shows cavitation induced by application of high-intensity focused ultrasound as observed by diagnostic ultrasound in an abscess induced in a rabbit model.

FIG. 8 is an image of a rabbit model showing no thermal injury after treatment with cavitation HIFU therapy.

FIGS. 9A and 9B show data derived from PCD measurements in which a 1.1 MHz HIFU source was used to treat mouse pancreatic tumors in vivo or in vitro at various pressure amplitudes.

### DETAILED DESCRIPTION

The present technology is generally directed to systems for delivering therapeutic acoustic energy (e.g., high-intensity focused ultrasound energy, referred to herein generally as “HIFU”) to an abscess or an infected fluid collection, and methods of treating an abscess or an infected fluid collection (e.g., reducing, ablating and/or eliminating an abscess or an infected fluid collection) using such systems. In some embodiments, the abscess or the infected fluid collection is an intra-abdominal abscess. In other embodiments, the abscess or the infected fluid collection is within the pelvic cavity of a subject. In still further embodiments, the abscess or infected fluid collection may be at other locations on the subject. Systems and methods configured in accordance with embodiments of the present technology are expected to provide effective non-surgical (e.g., non-invasive) treatment of abscesses or infected fluid collections while reducing risks commonly associated with conventional methods such as drainage.

HIFU is based on the same general principles as diagnostic ultrasound, but differs in the intensity of the acoustic waves. For example, both typical diagnostic ultrasound and HIFU may include sound waves with frequencies ranging from about 0.1 MHz to about 50 MHz. However, HIFU waves applied according to embodiments of the present technology may include a peak pressure ranging from about −1000 MPa to about 1000 MPa, pressures many orders of magnitude higher than ultrasound waves used for diagnostic purposes.

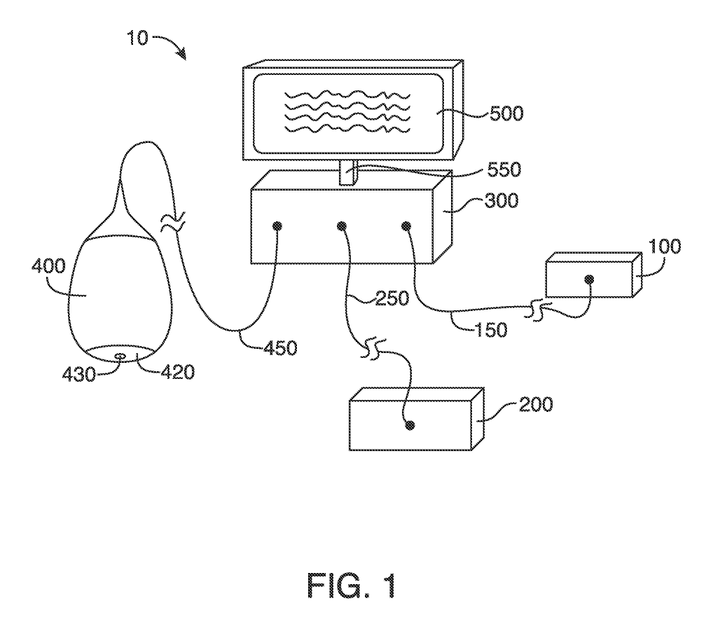

Therapeutic systems configured in accordance with embodiments of the present technology are configured to apply HIFU waves to an abscess or an infected fluid collection. In some embodiments, therapeutic acoustic energy generated by a system configured according to the present technology or applied to an abscess or an infected fluid collection according to methods of the present technology consists of HIFU. In some embodiments, therapeutic acoustic energy generated by a system configured according to the present technology or applied to an abscess or an infected fluid collection according to methods of the present technology consists essentially of HIFU. In some embodiments, therapeutic acoustic energy generated by a system configured according to the present technology or applied to an abscess or an infected fluid collection according to methods of the present technology comprises HIFU. In some embodiments, the therapeutic acoustic energy consists of HIFU energy Non-target tissue surrounding the abscess or an infected fluid collection may be generally unaffected (e.g., not damaged or not significantly damaged) by the HIFU waves. Methods of treating an abscess or an infected fluid collection according to embodiments of the present technology may be used non-invasively, for example without draining or aspirating the abscess or an infected fluid collection, reducing risks commonly associated with invasive abscess or infected fluid collection treatment techniques. Specific details of several embodiments of the present technology are described herein with reference to FIGS. 1-14B. Although many of the embodiments are described herein with respect to treating an abscess or an infected fluid collection, other applications and other embodiments in addition to those described herein are within the scope of the present technology. For example, some embodiments may be useful treating an abscess or an infected fluid collection in a subject in conjunction with another therapy, such as percutaneous catheter drainage, antibiotic drug therapy and/or antifungal drug therapy. Moreover, a person of ordinary skill in the art will understand that embodiments of the present technology can have components and/or procedures in addition to those shown or described herein, and that these and other embodiments can be without several of the components and/or procedures shown or described herein without deviating from the present technology. The headings provided herein are for convenience only.

In one embodiment, the present technology provides a method of treating an abscess or an infected fluid collection associated with a subject, the method comprising applying therapeutic acoustic energy to the abscess or infected fluid collection sufficient to disrupt and/or destroy at least a portion of a pathogenic component of the abscess or the infected fluid collection. In some embodiments, the therapeutic acoustic energy comprises HIFU. In some embodiments, the therapeutic acoustic energy increases the temperature of the abscess or the infected fluid collection substantially only at and in close proximity to the focal point (the “focal area”) of the therapeutic acoustic energy (e.g., histotripsy). In other embodiments, the therapeutic acoustic energy increases the temperature of the abscess or the infected fluid collection at the focal point of the therapeutic acoustic energy and in tissue and/or fluid surrounding the focal area (e.g., focused ultrasound (“FUS”) therapy).

In some embodiments, the present technology provides a method of inducing cavitation in an abscess or an infected fluid collection using, for example, HIFU energy focused within at least a portion of the abscess or the infected fluid collection.

The present technology may also include an acoustic abscess ablation system comprising a signal generator configured to generate an ultrasound waveform, an amplifier in operative communication with the signal generator for converting the ultrasound waveform into the high-intensity ultrasound waveform having an increased intensity to a signal transducer, and a signal transducer having an adjustable focus for delivering the HIFU energy to an abscess. In some embodiments, the abscess ablation system is used to treat an infected fluid collection instead of or in addition to an abscess.

For ease of reference, throughout this disclosure identical reference numbers are used to identify similar or analogous components or features, but the use of the same reference number does not imply that the parts should be construed to be identical. Indeed, in many examples described herein, the identically-numbered parts are distinct in structure and/or function.

For ease of reference, throughout this disclosure systems and methods of treatment may refer to “an abscess.” Unless the context clearly dictates otherwise, the term “an abscess” may refer to an abscess, an infected fluid collection, a septated abscess, a septated infected fluid collection, more than one abscess, more than one infected fluid collection, or a combination thereof.

Generally, unless the context indicates otherwise, the terms “distal” and “proximal” within this disclosure reference a position or direction with respect to the treating clinician or clinician's therapeutic tool (e.g., an ultrasound transducer). “Distal” or “distally” are a position distant from or in a direction away from the clinician or clinician's therapeutic tool. “Proximal” and “proximally” are a position near or in a direction toward the clinician or clinician's therapeutic tool.

### Selected Embodiments of Acoustic Abscess Ablation Systems and Methods

FIG. 1 is a partially schematic view of an acoustic abscess ablation system 10 (“system 10”) configured to administer therapeutic acoustic energy to an abscess of a patient or subject in accordance with an embodiment of the present technology. The system 10 in the embodiment of FIG. 1 comprises a signal generator 100 configured to generate an acoustic energy waveform, such as an ultrasound waveform and an amplifier 200 in operative communication with the signal generator 100. In some embodiments, the amplifier 200 is configured to convert the ultrasound waveform from the signal generator 100 into a high-intensity ultrasound waveform having an increased intensity. The signal generator 100 may be coupled with one or more components of system 10 using, for example, one or more wires 150.

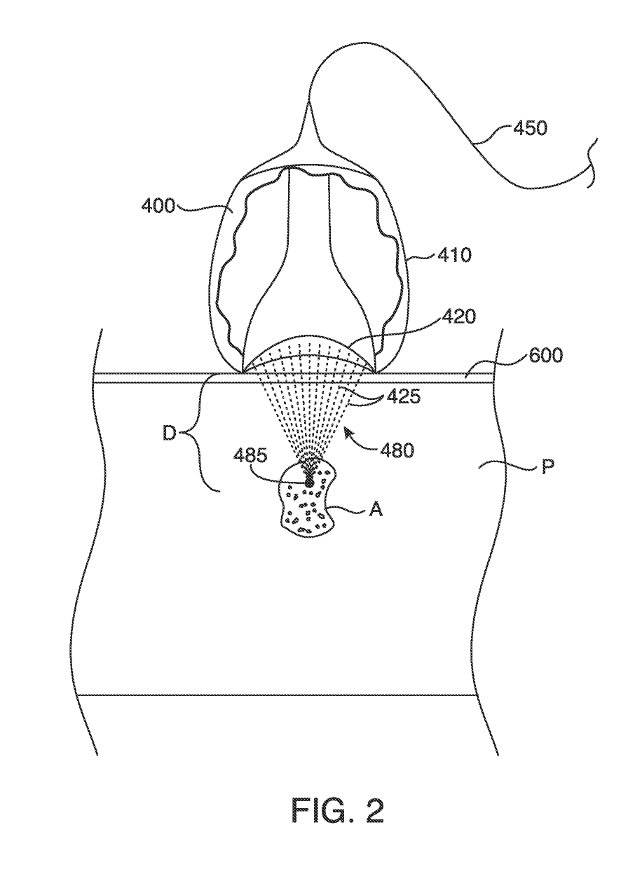

The system 10 also includes a transducer 400 configured for delivering the high-intensity ultrasound waveform to the target abscess of the patient (see FIG. 2). In some embodiments, the system 10 further comprises a second transducer 430 for obtaining an ultrasound image of the abscess. The second transducer 430 (when present) may comprise, for example, a diagnostic ultrasound transducer. Although the first transducer 400 and second transducer 430 are shown in FIG. 1 within a single transducer housing (ultrasound wand), one of skill in the art will recognize that the second transducer 430 may also be housed in a separate transducer housing from the first transducer 400 in other embodiments.

The system 100 may also comprise an optional controller 300 in operative communication with the signal generator 100, the amplifier 200 and the transducer 400, and an optional display 500 in operative communication with the signal generator 100, the amplifier 200, the transducer 400 and the optional controller 300.

The ultrasound waveform generated via the signal generator 100 may include a single frequency of ultrasound energy or more than one frequency of ultrasound energy. In some embodiments, for example, the ultrasound waveform comprises an ultrasound frequency of about 0.1 MHz to about 100 MHz, about 1 MHz to about 10 MHz, or about 1 MHz to about 5 MHz, for example about 0.1 MHz, about 0.2 MHz, about 0.3 MHz, about 0.4 MHz, about 0.5 MHz, about 0.6 MHz, about 0.7 MHz, about 0.8 MHz, about 0.9 MHz, about 1 MHz, about 2 MHz, about 3 MHz, about 4 MHz, about 5 MHz, about 6 MHz, about 7 MHz, about 8 MHz, about 9 MHz, about 10 MHz, about 11 MHz, about 12 MHz, about 13 MHz, about 14 MHz, about 15 MHz, about 16 MHz, about 17 MHz, about 18 MHz, about 19 MHz, about 20 MHz, about 21 MHz, about 22 MHz, about 23 MHz, about 24 MHz, about 25 MHz, about 26 MHz, about 27 MHz, about 28 MHz, about 29 MHz, about 30 MHz, about 31 MHz, about 32 MHz, about 33 MHz, about 34 MHz, about 35 MHz, about 36 MHz, about 37 MHz, about 38 MHz, about 39 MHz, about 40 MHz, about 41 MHz, about 42 MHz, about 43 MHz, about 44 MHz, about 45 MHz, about 46 MHz, about 47 MHz, about 48 MHz, about 49 MHz, about 50 MHz, about 51 MHz, about 52 MHz, about 53 MHz, about 54 MHz, about 55 MHz, about 56 MHz, about 57 MHz, about 58 MHz, about 59 MHz, about 60 MHz, about 61 MHz, about 62 MHz, about 63 MHz, about 64 MHz, about 65 MHz, about 66 MHz, about 67 MHz, about 68 MHz, about 69 MHz, about 70 MHz, about 71 MHz, about 72 MHz, about 73 MHz, about 74 MHz, about 75 MHz, about 76 MHz, about 77 MHz, about 78 MHz, about 79 MHz, about 80 MHz, about 81 MHz, about 82 MHz, about 83 MHz, about 84 MHz, about 85 MHz, about 86 MHz, about 87 MHz, about 88 MHz, about 89 MHz, about 90 MHz, about 91 MHz, about 92 MHz, about 93 MHz, about 94 MHz, about 95 MHz, about 96 MHz, about 97 MHz, about 98 MHz, about 99 MHz, about 100 MHz, or greater than about 100 MHz. In some embodiments, the frequency is selected to provide a focal area that is smaller than the size of the abscess to be treated and/or to provide an attenuation (e.g., depth) sufficient to contact the abscess with the ultrasound energy.

The signal generator 100 is also configured to provide a specific form (e.g., shape, pulse pattern, etc.) to the acoustic energy waveform. In some embodiments, for example, the signal generator 100 is configured to provide an acoustic energy waveform comprising periodic pulse sequences, non-periodic pulse sequences, or a combination thereof. For example, in some embodiments the signal generator 100 is configured to provide an acoustic energy waveform comprising a sinusoidal waveform. In other embodiments, the signal generator 100 is configured to provide an acoustic energy waveform comprising a square waveform. In still further embodiments, the signal generator 100 is configured to provide an acoustic energy waveform comprising a peaked waveform. In yet other embodiments, the signal generator 100 may be configured to provide an acoustic energy waveform comprising a combination of any of the foregoing.

As noted above, the amplifier 200 is in operative communication with the signal generator 100 (e.g., via one or more wires 250) and is configured to amplify the acoustic energy waveform generated by the signal generator 100.

In some embodiments, for example, the amplifier 200 outputs an amplified acoustic energy waveform having a peak negative pressure sufficient to induce cavitation of the abscess. In some embodiments, the amplifier 200 outputs an amplified acoustic energy waveform having a peak negative pressure of at least about −0.1 MPa, for example at least about −0.1 MPa, at least about −0.2 MPa, at least about −0.3 MPa, at least about −0.4 MPa, at least about −0.5 MPa, at least about −0.6 MPa, at least about −0.7 MPa, at least about −0.8 MPa, at least about −0.9 MPa, at least about −1 MPa, at least about −2 MPa, at least about −3 MPa, at least about −4 MPa, at least about −5 MPa, at least about −6 MPa, at least about −7 MPa, at least about −8 MPa, at least about −9 MPa, at least about −10 MPa, at least about −11 MPa, at least about −12 MPa, at least about −13 MPa, at least about −14 MPa, at least about −15 MPa, at least about −16 MPa, at least about −17 MPa, at least about −18 MPa, at least about −19 MPa, or at least about −20 MPa. In some embodiments, the amplifier 200 outputs an amplified acoustic energy waveform having a peak negative pressure determined based on the location, size, and/or characteristics of the abscess.

In some embodiments, the amplifier 200 outputs an amplified acoustic energy waveform having a peak positive pressure of at least about 0.1 MPa, for example −1000 MPa to about 1000 MPa, for example about 0.1 MPa, at least about 0.2 MPa, at least about 0.3 MPa, at least about 0.4 MPa, at least about 0.5 MPa, at least about 0.6 MPa, at least about 0.7 MPa, at least about 0.8 MPa, at least about 0.9 MPa, at least about 1 MPa, at least about 2 MPa, at least about 3 MPa, at least about 4 MPa, at least about 5 MPa, at least about 6 MPa, at least about 7 MPa, at least about 8 MPa, at least about 9 MPa, at least about 10 MPa, at least about 11 MPa, at least about 12 MPa, at least about 13 MPa, at least about 14 MPa, at least about 15 MPa, at least about 16 MPa, at least about 17 MPa, at least about 18 MPa, at least about 19 MPa, or at least about 20 MPa, at least about 25 MPa, about 50 MPa, about 75 MPa, about 100 MPa, about 125 MPa, about 150 MPa, about 175 MPa, about 200 MPa, about 225 MPa, about 250 MPa, about 275 MPa, about 300 MPa, about 325 MPa, about 350 MPa, about 375 MPa, about 400 MPa, about 425 MPa, about 450 MPa, about 475 MPa, about 500 MPa, about 525 MPa, about 550 MPa, about 575 MPa, about 600 MPa, about 625 MPa, about 650 MPa, about 675 MPa, about 700 MPa, about 725 MPa, about 750 MPa, about 775 MPa, about 800 MPa, about 825 MPa, about 850 MPa, about 875 MPa, about 900 MPa, about 925 MPa, about 950 MPa, about 975 MPa, or about 1000 MPa.

In some embodiments, the amplifier 200 outputs an amplified acoustic energy waveform having a time-averaged intensity of about 50 W/cm² to about 20,000 W/cm² at the focus, for example about 50 W/cm², about 100 W/cm², about 150 W/cm², about 200 W/cm², about 250 W/cm², about 300 W/cm², about 350 W/cm², about 400 W/cm², about 450 W/cm², about 500 W/cm², about 550 W/cm², about 600 W/cm², about 650 W/cm², about 700 W/cm², about 750 W/cm², about 800 W/cm², about 850 W/cm², about 900 W/cm², about 950 W/cm², about 1000 W/cm², about 1100 W/cm², about 1200 W/cm², about 1300 W/cm², about 1400 W/cm², about 1500 W/cm², about 1600 W/cm², about 1700 W/cm², about 1800 W/cm², about 1900 W/cm², about 2000 W/cm², about 2100 W/cm², about 2200 W/cm², about 2300 W/cm², about 2400 W/cm², about 2500 W/cm², about 2600 W/cm², about 2700 W/cm², about 2800 W/cm², about 2900 W/cm², about 3000 W/cm², about 3100 W/cm², about 3200 W/cm², about 3300 W/cm², about 3400 W/cm², about 3500 W/cm², about 3600 W/cm², about 3700 W/cm², about 3800 W/cm², about 3900 W/cm², about 4000 W/cm², about 4100 W/cm², about 4200 W/cm², about 4300 W/cm², about 4400 W/cm², about 4500 W/cm², about 4600 W/cm², about 4700 W/cm², about 4800 W/cm², about 4900 W/cm², about 5000 W/cm², about 5100 W/cm², about 5200 W/cm², about 5300 W/cm², about 5400 W/cm², about 5500 W/cm², about 5600 W/cm², about 5700 W/cm², about 5800 W/cm², about 5900 W/cm², about 6000 W/cm², about 6100 W/cm², about 6200 W/cm², about 6300 W/cm², about 6400 W/cm², about 6500 W/cm², about 6600 W/cm², about 6700 W/cm², about 6800 W/cm², about 6900 W/cm², about 7000 W/cm², about 7100 W/cm², about 7200 W/cm², about 7300 W/cm², about 7400 W/cm², about 7500 W/cm², about 7600 W/cm², about 7700 W/cm², about 7800 W/cm², about 7900 W/cm², about 8000 W/cm², about 8100 W/cm², about 8200 W/cm², about 8300 W/cm², about 8400 W/cm², about 8500 W/cm², about 8600 W/cm², about 8700 W/cm², about 8800 W/cm², about 8900 W/cm², about 9000 W/cm², about 9100 W/cm², about 9200 W/cm², about 9300 W/cm², about 9400 W/cm², about 9500 W/cm², about 9600 W/cm², about 9700 W/cm², about 9800 W/cm², about 9900 W/cm², or about 10,000 W/cm² at the focus.

In some embodiments, the amplifier 200 outputs an amplified acoustic energy waveform having a peak compression pressure of up to about 30 MPa, for example about 1 MPa, about 2 MPa, about 3 MPa, about 4 MPa, about 5 MPa, about 6 MPa, about 7 MPa, about 8 MPa, about 9 MPa, about 10 MPa, about 11 MPa, about 12 MPa, about 13 MPa, about 14 MPa, about 15 MPa, about 16 MPa, about 17 MPa, about 18 MPa, about 19 MPa, about 20 MPa, about 21 MPa, about 22 MPa, about 23 MPa, about 24 MPa, about 25 MPa, about 26 MPa, about 27 MPa, about 28 MPa, about 29 MPa, or about 30 MPa.

In some embodiments, the amplifier 200 outputs an amplified acoustic energy waveform having a peak rarefaction pressure of up to about 10 MPa, for example about 0.1 MPa, about 0.2 MPa, about 0.3 MPa, about 0.4 MPa, about 0.5 MPa, about 0.6 MPa, about 0.7 MPa, about 0.8 MPa, about 0.9 MPa, about 1 MPa, about 2 MPa, about 3 MPa, about 4 MPa, about 5 MPa, about 6 MPa, about 7 MPa, about 8 MPa, about 9 MPa, or about 10 MPa.

In other embodiments, however, the amplifier 200 may be configured to output an amplified acoustic energy waveform having more than one peak pressure.

The transducer 400 is operatively coupled with one or more components of the system 10 (e.g., via one or more wires 450) and is configured to administer (e.g., apply, deliver, etc.) the amplified acoustic energy waveform to an abscess. In some embodiments, the transducer 400 includes a transducer head 420 configured to focus the amplified acoustic energy waveform within an abscess of a subject. In some embodiments, the transducer 400 may additionally include a second transducer head 430 for obtaining an image of the abscess or infected fluid collection for monitoring treatment. In other embodiments, the second transducer head 430 is housed in a second, separate transducer.

The transducer head 420 is arranged to provide the amplified acoustic energy waveform with an adjustable focus. In some embodiments, for example, the transducer head 420 includes an array of transducer elements 425, each of which may be energized in a pattern sufficient to provide the amplified acoustic energy waveform having a focus located at a preselected distance from the transducer 400. FIG. 2, for example, is a partially schematic view of a method of applying HIFU energy to an abscess using the system 10. As shown in the embodiment of FIG. 2, the transducer head 420 has a generally concave shape, providing an array of transducer elements 425 in a generally concave pattern. The resulting amplified acoustic energy waveform 480 provided by the transducer 400 includes a focal point 485 located a predetermined focal distance D from the transducer 400. The focal distance D may be selected to correspond to the location (e.g., depth) of an abscess A such that the focal point 485 is located within the abscess A of a patient P. One of skill in the art will recognize, however, that other arrangements of transducer elements 425 are possible. For example, linear arrays of transducer elements 425 or transducer elements 425 arranged in other suitable patterns may also be used. In addition, the focal distance D may be determined or adjusted by altering the phasing of the amplified acoustic energy waveform provided to each of the transducer elements 425.

Referring to FIGS. 1 and 2 together and as noted previously, in some embodiments the system 10 may include a controller 300 in operative communication with the signal generator 100, the amplifier 200 and the transducer 400. The controller 300 may be used, for example, to receive input from a clinician regarding the focal distance D required, the extent of amplification required by the amplifier 200, and/or the shape of the acoustic energy waveform generated by the signal generator 100. In some embodiments, the controller 300 is configured to receive data (e.g., imaging data) from a second transducer 430 and determine a focal distance D based on the data. The controller may also be configured to automatically determine and/or automatically adjust the extent of amplification provided by the amplifier, the shape of the waveform provided by the signal generator 100, and/or a pattern of energizing one or more transducer elements 425 as a function of imaging data showing the abscess A to be treated (e.g., including a depth of the abscess A below the surface of the patient P's skin.

In some embodiments, the system 10 further includes a display 500 (in operative communication with the controller 300) configured to display information about the therapy. The display 500 can include, for example, a user interface (e.g., a graphical user interface or GUI) enabling a user (e.g., a clinician) to input parameters and/or patient data corresponding to the therapy. In some embodiments, the display 500 is configured to display a diagnostic image and/or a real-time image comprising at least a portion of the abscess to be treated.

In any of the embodiments disclosed herein, the system 10 may be configured to be portable. In such embodiments, the system 10 may further include a battery for providing power to the system 10.

Methods of treating an abscess (e.g., ablating an abscess) may include the use of the systems and/or components described herein (such as the system 10 described above with reference to FIGS. 1 and 2) or other suitable systems. In some embodiments, for example, the present technology provides a method of treating an abscess associated with a subject comprising applying therapeutic acoustic energy to the abscess sufficient to disrupt and/or destroy at least a portion of a pathogenic component of the abscess. The pathogen may include one or more types of bacteria, one or more types of fungi, one or more strains of virus, or a combination thereof

In some embodiments, the abscess is associated with a disease or disorder, such as appendicitis, pancreatitis, cholecystectomy or gallbladder perforations, biliary leakage, gastrointestinal perforations, enteric fistulas, hernias and volvulus, diverticulitis, intussusceptions, post-operative infections or leakages, malignancies, ischemia and embolic disease, vasculitis, trauma, local radiation therapy or brachytherapy, a localized infection or abscess within a solid organ, a localized infection or abscess within a subcutaneous soft tissue, cystitis, pyelonephritis, urethritis, prostatitis, a genitourinary abscess, an infection after surgical revisions such as a diverting ileostomy, a neobladder, an infection associated with a nephrostomy stent/drain, an infection associated with a suprapubic drains, an infection associated with a stone, an infection associated with a malignancy, an infection associated with a trauma, an infection associated with a fistulas, an infection associated with a bladder/ureteric perforation, acute appendicitis, acute cholecystitis, mastitis, cellulitis, erysipelas, a thermal burn, a chemical burn, a breast malignancy, a skin malignancy, lymphoma, a mycobacterium, an allergic reaction, a lymphatic obstruction, a surgery, a biopsy, a piercing, a tattoo, a venous obstruction, an abscess within a rectus abdominis muscle, an abscess within a transverse abdominis muscle, an abscess within a psoas muscle, an abscess within a levator ani muscle, an abscess within a piriformis muscle, an abscess within an obturator muscle, an abscess within an adductor muscle, an abscess within a gluteal muscle, an abscess within a muscle of a shoulder girdle, an abscess within a rotator cuff, an abscess within a chest wall, an abscess within a pectus muscle, an abscess within a serratus muscle, an abscess within a sternocleidomastoid muscle, an abscess within a latissimus dorsi muscle, an abscess within a trapezius muscle, an abscess within a biceps muscle, an abscess within a triceps brachii muscle, an abscess within a deltoid muscle, parapneumonic effusion, empyema, chylothorax, pericarditis, mediastinitis, or a combination thereof

Abscesses generally contain a fluid component. Unlike solid tissues, which must be fully exposed to a radiative therapy (usually with a margin), the fluidic properties of abscesses enable effective treatment of the entire contents of the abscess by exposing only a portion of the abscess to therapeutic acoustic energy (e.g., HIFU). In some embodiments, the therapeutic acoustic energy is focused on only a portion of the abscess. In other embodiments, however, methods for treating an abscess may comprise focusing therapeutic acoustic energy on more than one location within the abscess. In still other embodiments, the therapeutic acoustic energy is focused on all or substantially all of the abscess.

In some embodiments, the therapeutic acoustic energy comprises a continuous wave of acoustic energy. In other embodiments, however, the therapeutic acoustic energy comprises a plurality of energy pulses. In such embodiments, the pulses may be administered at a single pulse repetition frequency (“PRF”) or at more than one PRF. Lower PRF values correlate to longer treatment times, while higher PRF values provide shorter treatment times. In some embodiments, the PRF is about 1 kHz to about 1 MHz, for example about 1 kHz, about 2 kHz, about 3 kHz, about 4 kHz, about 5 kHz, about 6 kHz, about 7 kHz, about 8 kHz, about 9 kHz, about 10 kHz, about 15 kHz, about 20 kHz, about 25 kHz, about 30 kHz, about 35 kHz, about 40 kHz, about 45 kHz, about 50 kHz, about 55 kHz, about 60 kHz, about 65 kHz, about 70 kHz, about 75 kHz, about 80 kHz, about 85 kHz, about 90 kHz, about 95 kHz, about 100 kHz, about 125 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 525 kHz, about 550 kHz, about 575 kHz, about 600 kHz, about 625 kHz, about 650 kHz, about 675 kHz, about 700 kHz, about 725 kHz, about 750 kHz, about 775 kHz, about 800 kHz, about 825 kHz, about 850 kHz, about 875 kHz, about 900 kHz, about 925 kHz, about 950 kHz, about 975 kHz, or about 1 MHz.

The therapeutic acoustic energy is applied to target tissue for a time sufficient to effectively treat an abscess. In some embodiments, the therapeutic acoustic energy is applied for about 30 seconds to about 15 minutes, or about 5 minutes to about 12 minutes, for example about 30 seconds, about 1 minute, about 1.1 minutes, about 1.2 minutes, about 1.3 minutes, about 1.4 minutes, about 1.5 minutes, about 1.6 minutes, about 1.7 minutes, about 1.8 minutes, about 1.9 minutes, about 2 minutes, about 2.1 minutes, about 2.2 minutes, about 2.3 minutes, about 2.4 minutes, about 2.5 minutes, about 2.6 minutes, about 2.7 minutes, about 2.8 minutes, about 2.9 minutes, about 3 minutes, about 3.1 minutes, about 3.2 minutes, about 3.3 minutes, about 3.4 minutes, about 3.5 minutes, about 3.6 minutes, about 3.7 minutes, about 3.8 minutes, about 3.9 minutes, about 4 minutes, about 4.1 minutes, about 4.2 minutes, about 4.3 minutes, about 4.4 minutes, about 4.5 minutes, about 4.6 minutes, about 4.7 minutes, about 4.8 minutes, about 4.9 minutes, about 5 minutes, about 5.1 minutes, about 5.2 minutes, about 5.3 minutes, about 5.4 minutes, about 5.5 minutes, about 5.6 minutes, about 5.7 minutes, about 5.8 minutes, about 5.9 minutes, about 6 minutes, about 6.1 minutes, about 6.2 minutes, about 6.3 minutes, about 6.4 minutes, about 6.5 minutes, about 6.6 minutes, about 6.7 minutes, about 6.8 minutes, about 6.9 minutes, about 7 minutes, about 7.1 minutes, about 7.2 minutes, about 7.3 minutes, about 7.4 minutes, about 7.5 minutes, about 7.6 minutes, about 7.7 minutes, about 7.8 minutes, about 7.9 minutes, about 8 minutes, about 8.1 minutes, about 8.2 minutes, about 8.3 minutes, about 8.4 minutes, about 8.5 minutes, about 8.6 minutes, about 8.7 minutes, about 8.8 minutes, about 8.9 minutes, about 9 minutes, about 9.1 minutes, about 9.2 minutes, about 9.3 minutes, about 9.4 minutes, about 9.5 minutes, about 9.6 minutes, about 9.7 minutes, about 9.8 minutes, about 9.9 minutes, about 10 minutes, about 10.1 minutes, about 10.2 minutes, about 10.3 minutes, about 10.4 minutes, about 10.5 minutes, about 10.6 minutes, about 10.7 minutes, about 10.8 minutes, about 10.9 minutes, about 11 minutes, about 11.1 minutes, about 11.2 minutes, about 11.3 minutes, about 11.4 minutes, about 11.5 minutes, about 11.6 minutes, about 11.7 minutes, about 11.8 minutes, about 11.9 minutes, about 12 minutes, about 12.1 minutes, about 12.2 minutes, about 12.3 minutes, about 12.4 minutes, about 12.5 minutes, about 12.6 minutes, about 12.7 minutes, about 12.8 minutes, about 12.9 minutes, about 13 minutes, about 13.1 minutes, about 13.2 minutes, about 13.3 minutes, about 13.4 minutes, about 13.5 minutes, about 13.6 minutes, about 13.7 minutes, about 13.8 minutes, about 13.9 minutes, about 14 minutes, about 14.1 minutes, about 14.2 minutes, about 14.3 minutes, about 14.4 minutes, about 14.5 minutes, about 14.6 minutes, about 14.7 minutes, about 14.8 minutes, about 14.9 minutes, or about 15 minutes. In some embodiments, the therapeutic acoustic energy is applied to target tissue for about 30 seconds to about 15 minutes or about 5 minutes to about 12 minutes for more than one iteration, for example in some embodiments the methods may include serially treating the abscess at least twice, at least thrice, at least four times, five times, at least six times, at least seven times, at least eight times, at least nine times, or at least ten times.

In some embodiments, the pulses are administered for durations and at intervals (e.g., PRF) in order to provide a relatively low duty factor. Without wishing to be bound by theory, it is believed that lower duty factor HIFU therapy prevents surrounding tissue temperatures from increasing to an extent where damage occurs. In some embodiments, the therapy is administered to provide a duty factor of about 0.1% to about 10%, about 0.1% to about 5%, about 0.5% to about 4%, or about 1% to about 3%, for example about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, or about 10%.

Methods of the present technology may comprise administering HIFU to the abscess. In such embodiments, the HIFU may be applied to have a peak pressure comprising about −20 MPa to about 1000 MPa, for example about −20 MPa, about 19 MPa, about −18 MPa, about −17 MPa, about −16 MPa, about −15 MPa, about −14 MPa, about −13 MPa, about −12 MPa, about −11 MPa, about −10 MPa, about −9 MPa, about −8 MPa, about −7 MPa, about −6 MPa, about −5 MPa, about −4 MPa, about −3 MPa, about −2 MPa, about −1 MPa, about 0 MPa, about 50 MPa, about 100 MPa, about 150 MPa, about 200 MPa, about 250 MPa, about 300 MPa, about 350 MPa, about 400 MPa, about 450 MPa, about 500 MPa, about 550 MPa, about 600 MPa, about 650 MPa, about 700 MPa, about 750 MPa, about 800 MPa, about 850 MPa, about 900 MPa, about 950 MPa, and/or about 1000 MPa. In some embodiments, the HIFU is administered to have a rarefaction peak pressure of about −20 MPa to about −1 MPa, for example about −20 MPa, about 19 MPa, about −18 MPa, about −17 MPa, about −16 MPa, about −15 MPa, about −14 MPa, about −13 MPa, about −12 MPa, about −11 MPa, about −10 MPa, about −9 MPa, about −8 MPa, about −7 MPa, about −6 MPa, about −5 MPa, about −4 MPa, about −3 MPa, about −2 MPa, or about −1 MPa. The HIFU energy may also be applied to have a compression peak pressure of about 1 MPa to about 1000 MPa, for example about 1 MPa, about 2 MPa, about 3 MPa, about 4 MPa, about 5 MPa, about 6 MPa, about 7 MPa, about 8 MPa, about 9 MPa, about 10 MPa, about 20 MPa, about 30 MPa, about 40 MPa, about 50 MPa, about 60 MPa, about 70 MPa, about 80 MPa, about 90 MPa, about 100 MPa, about 110 MPa, about 120 MPa, about 130 MPa, about 140 MPa, about 150 MPa, about 160 MPa, about 170 MPa, about 180 MPa, about 190 MPa, about 200 MPa, about 210 MPa, about 220 MPa, about 230 MPa, about 240 MPa, about 250 MPa, about 260 MPa, about 270 MPa, about 280 MPa, about 290 MPa, about 300 MPa, about 310 MPa, about 320 MPa, about 330 MPa, about 340 MPa, about 350 MPa, about 360 MPa, about 370 MPa, about 380 MPa, about 390 MPa, about 400 MPa, about 410 MPa, about 420 MPa, about 430 MPa, about 440 MPa, about 450 MPa, about 460 MPa, about 470 MPa, about 480 MPa, about 490 MPa, about 500 MPa, about 510 MPa, about 520 MPa, about 530 MPa, about 540 MPa, about 550 MPa, about 560 MPa, about 570 MPa, about 580 MPa, about 590 MPa, about 600 MPa, about 610 MPa, about 620 MPa, about 630 MPa, about 640 MPa, about 650 MPa, about 660 MPa, about 670 MPa, about 680 MPa, about 690 MPa, about 700 MPa, about 710 MPa, about 720 MPa, about 730 MPa, about 740 MPa, about 750 MPa, about 760 MPa, about 770 MPa, about 780 MPa, about 790 MPa, about 800 MPa, about 810 MPa, about 820 MPa, about 830 MPa, about 840 MPa, about 850 MPa, about 860 MPa, about 870 MPa, about 880 MPa, about 890 MPa, about 900 MPa, about 910 MPa, about 920 MPa, about 930 MPa, about 940 MPa, about 950 MPa, about 960 MPa, about 970 MPa, about 980 MPa, about 990 MPa, or about 1000 MPa.

In some embodiments, the therapeutic acoustic energy is applied transcutaneously, for example using an ultrasound wand designed to be used externally and without causing an incision or other breach of the subject's skin. In some embodiments, the therapeutic acoustic energy is applied externally using an external ultrasound wand.

In other embodiments, the therapeutic acoustic energy is applied internally, for example using an ultrasound transducer sized and shaped to be used internally. In such embodiments, the therapeutic acoustic energy may be applied using any ultrasound wand suitable for internal use, such as a transvaginal ultrasound wand (also referred to as a pelvic ultrasound wand) or on a catheter drainage system.

In some embodiments, a method of the present technology comprises applying therapeutic acoustic energy (e.g., HIFU) to an abscess in an amount sufficient to reduce the viscosity of the abscess fluid, and draining the fluid. In some embodiments, the step of draining the fluid comprises inserting a catheter into the abscess. In some embodiments, the therapeutic acoustic energy is applied before insertion of a catheter. In other embodiments, the therapeutic acoustic energy is applied after insertion of a catheter.

In some embodiments, the methods of the present technology further comprise targeting and/or tracking the abscess before applying the therapeutic acoustic energy to the abscess. In some embodiments, the methods of the present technology further comprise targeting and/or tracking the abscess during application of the therapeutic acoustic energy to the abscess. In some embodiments, the methods of the present technology further comprise targeting and/or tracking the abscess after applying the therapeutic acoustic energy to the abscess. Targeting and/or tracking the abscess may be accomplished using any suitable method known to those of skill in the art including, for example, diagnostic ultrasound, CT and/or MRI. In some embodiments, the therapeutic acoustic energy is applied until a change in the abscess's diagnostic scan image is detected. In some embodiments, the focal point of the therapeutic acoustic energy is adjusted during application of the therapeutic acoustic energy in response to movement or other change in the abscess detected by the diagnostic ultrasound, CT and/or MRI. In some embodiments, the adjustment of the focal point of the therapeutic acoustic energy is automatic in response to movement or other change in the abscess detected by the diagnostic ultrasound, CT and/or MRI. In some embodiments, the focal point is adjusted spatially (e.g., moved in comparison to an initial location of the focal point). In some embodiments, the focal point is adjusted by modulating the energy output by one or more transducer elements 425 (e.g., beam steering). In addition or instead, the focal point may be adjusted by mechanically moving the transducer relative to the abscess, for example using one or more computer-controlled actuators operatively coupled to the transducer 400.

In some embodiments, a diagnostic monitor provides targeting features that enable a clinician to manually position the treatment transducer to apply the therapeutic acoustic energy to an abscess. In some embodiments, the targeting features comprise one or more markers displayed on a display (e.g., display 500). In some embodiments, the one or more markers are displayed on the display over an image of the abscess, for example obtained by diagnostic ultrasound, CT and/or MRI (e.g., real-time diagnostic ultrasound). In some embodiments, the targeting features enable targeting of the abscess (e.g., positioning of the focal area of the therapeutic acoustic energy) within the abscess without substantially affecting surrounding tissue.

In some embodiments, the methods of the present technology further comprise obtaining one or more images of the abscess before applying the therapeutic acoustic energy to the abscess. Such image(s) of the abscess may obtained, for example, via ultrasound, computed tomography, magnetic resonance imaging, or other suitable imaging techniques. In one particular embodiment, the image of the abscess is an ultrasound image obtained using an ultrasound transducer array configured to additionally generate the therapeutic acoustic energy.

Methods in accordance with the present technology are expected to reduce or eliminate the need for invasive therapy (e.g., draining the abscess, exploratory surgery, etc.) in patients. Further, methods of the present technology are also expected to reduce or eliminate the need for drug therapy, such as treatment of the abscess with an active agent (e.g., an antibiotic, an antifungal, and/or an antiviral agent).

In some embodiments, treatment using the methods described herein may induce cavitation in an abscess. The HIFU energy may be administered to the abscess in any manner described herein. Further, the HIFU energy may be applied in an amount sufficient to disrupt and/or destroy at least a portion of a pathogenic component of the abscess, such as a bacterium, a fungus, and/or a virus.

In some embodiments, the methods of the present technology further comprise applying the therapeutic acoustic energy to the abscess through a coupling medium. In some embodiments, the coupling medium is applied to the patient's skin before application of the therapeutic acoustic energy. Any suitable coupling medium (e.g., gel or liquid) may be used including, for example, ultrasound gel, water, a gel pad, saline and/or glycerol. In some embodiments, the coupling medium includes a wetting agent, for example to decrease the surface energy of the coupling medium, a gas reducing agent, for example to reduce solubilized gas and/or gas pockets within the coupling medium, or a combination thereof. In some embodiments, the coupling medium is applied after placing an adhesive drape onto the skin to contain the coupling medium within a predetermined area.

### EXAMPLES

### Example 1

### Acoustic Properties of Human Pus

Samples of human pus were obtained under IRB approval from regional hospitals associated with the University of Washington, along with identifier-free patient data describing the size, type, location, and microbiological findings. The attenuation coefficients and viscosities of the pus samples, including the frequency nonlinearity exponent and sound speed, were determined using an acoustic caliper bi-static device in which the sample was placed between one transmit and one receive transducer. Measurements were obtained at very low rates (Hz).

### Example 2

### In Vitro Treatment of Human Pus with HIFU

This example demonstrates that application of HIFU to in vitro human pus samples disrupts bacterial cell membranes.

Human pus samples were obtained from abscesses in randomly selected patients at each of two urban hospitals over a two-month period. The patients were either inpatients or emergency room patients. Each pus sample was confirmed by diagnostic imaging, drainage and microbiological characterizations.

The samples were obtained from varied etiologies (e.g., Crohn's disease, dismotility, post-operative, diverticulitis, fistula from ileostomy with inflammatory bowel disease, gastro-urinary/pelvic inflammatory disease/pyelonephrities, gunshot wound with bowel perforation, hemolytic-uremic syndrome/abdominal free fluid, infected CSFoma, lymphadenitis, pancreatitis, penetrating trauma, perianal fistula, polytrauma with bowel perforation, post-AAA stent complication, post-appendectomy, post-cholecystectomy leak, post-surgical, rectal cancer, ruptured appendicitis, ruptured cholecystitis, self-inflicted rectal trauma, spontaneous) and from various locations (e.g., gallbladder fossa, left lower anterior neck, left paraspinous, left perianal, left pericolic gutter, left piriformis muscle, left submandibular region, midline abdomen (iliac bifurcation), midline anterior abdomen, midline lower back superficial, midline pelvis, presacral, rectovesicular pouch/perirectal, retropharyngeal, retrovesicular pouch, right anterior abdominal compart, right lower quadrant, right paraspinous, right pelvis extending to presacral, right psoas, superficial abdominal wall, suprapubic, supravesicular). The average size of the abscesses was about 200 cm³ and ranged from about 7 cm³ to about 2000 cm³. About 90% of the abscesses were visible by ultrasound imaging. Most were in locations with a clear path accessible by ultrasound; some were adjacent to the patient's bowel and/or a solid organ. About ⅓ of the samples were from septated or multiloculated abscesses, and about ⅓ of the abscesses included internal gas.

Each of the pus samples was placed in a vacuum desiccator and degassed for 60 minutes to remove bubbles introduced by handling. The samples were then warmed to 37° C. and allowed to equilibrate with atmospheric gas supplemented with 5% CO₂ to simulate in vivo conditions. A 100-μL aliquot was withdrawn for viability testing according to standard methods well-known to a skilled artisan. The remainder was placed in a 37° C. bath and positioned at the focus of a HIFU source transducer operated at 2.5 MHz. A 5 MHz or 10 MHz focused transducer was used to detect characteristic cavitation noise; its focus was orthogonal to and overlapped with the HIFU focal zone (a standard passive cavitation setup). The HIFU burst length was fixed with short numbers of cycles (e.g., about 20 to about 50 cycles), and low pulse repetition frequencies (PRF). Thus there was no significant sample heating in these experiments, which was confirmed by thermocouple readings. The pressure amplitude was increased incrementally until the first burst of cavitation noise was observed; this threshold was defined as P1. Acoustic pressure was then increased until the first evidence of sustained cavitation (e.g., over most of the burst length) was observed; this threshold was defined as P2. When sustained cavitation was achieved, aliquots were taken for viability testing at intervals out to 10 minutes. The cumulated inertial cavitation dose developed during treatment of that sample was calculated and correlated with any observed reduction in microbial viability.

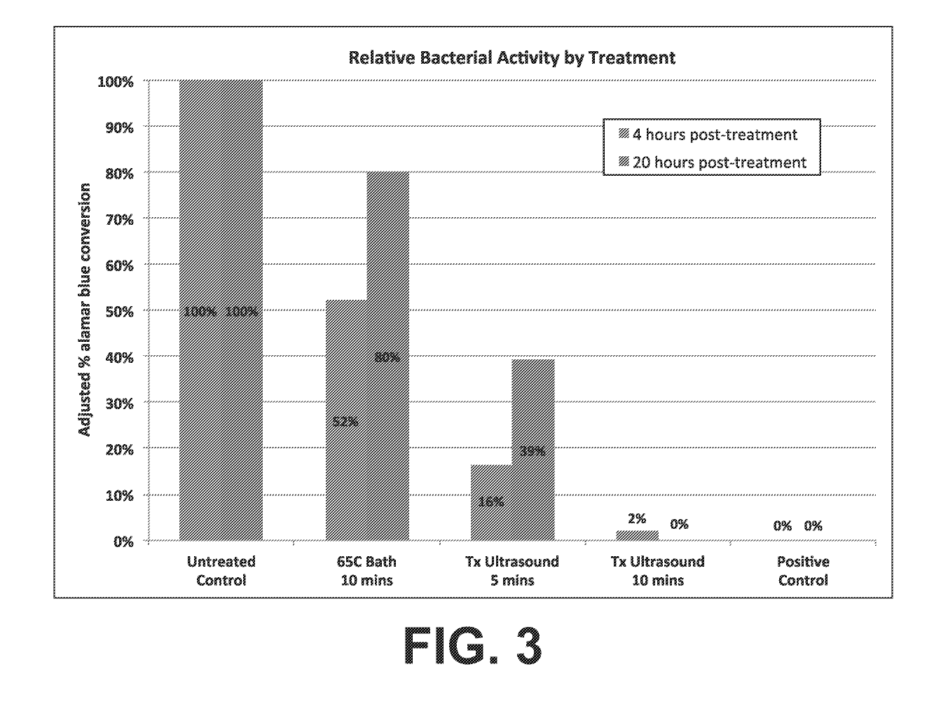

As shown in FIG. 3, treatment with HIFU (“Tx Ultrasound”) for 5 minutes dramatically reduced bacterial activity compared to untreated control and hot water bath. Even after 20 hours post-treatment, the bacterial activity was less than 40% of the untreated control, and less than half that of the hot water bath treatment. Treatment with HIFU for 10 minutes effectively destroyed the bacterial activity of the sample.

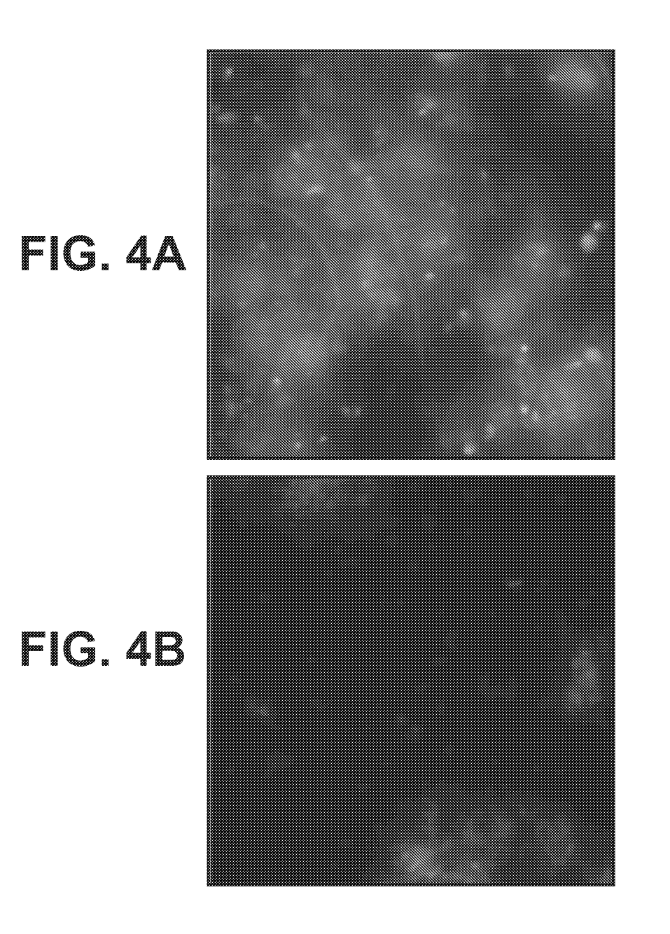

FIG. 4A shows differential immunofluorescent stained sample of human pus before in vitro treatment. After just 3 minutes of HIFU treatment, bacterial cell membranes were disputed and no intact bacteria were visible in the sample (FIG. 4B).

### Example 3

### In Vivo Treatment of Abscesses

A 4+ kg New Zealand white rabbit with unclear Pasteurella status was obtained and the bilateral thigh and the right paraspinous regions were trimmed using clippers with intact underlying skin. The animal had normal temperature and appearance. No initial labs were obtained. on Day 0 and under ultrasound imaging, the right thigh was injected with 0.5 cc of a solution of a combination of common gastrointestinal bacteria and barium, the left thigh was injected with 0.5 cc of a monomicrobial solution, and the right paraspinous region was injected with 0.5 cc of a sterile solution including 1% dextran (control).

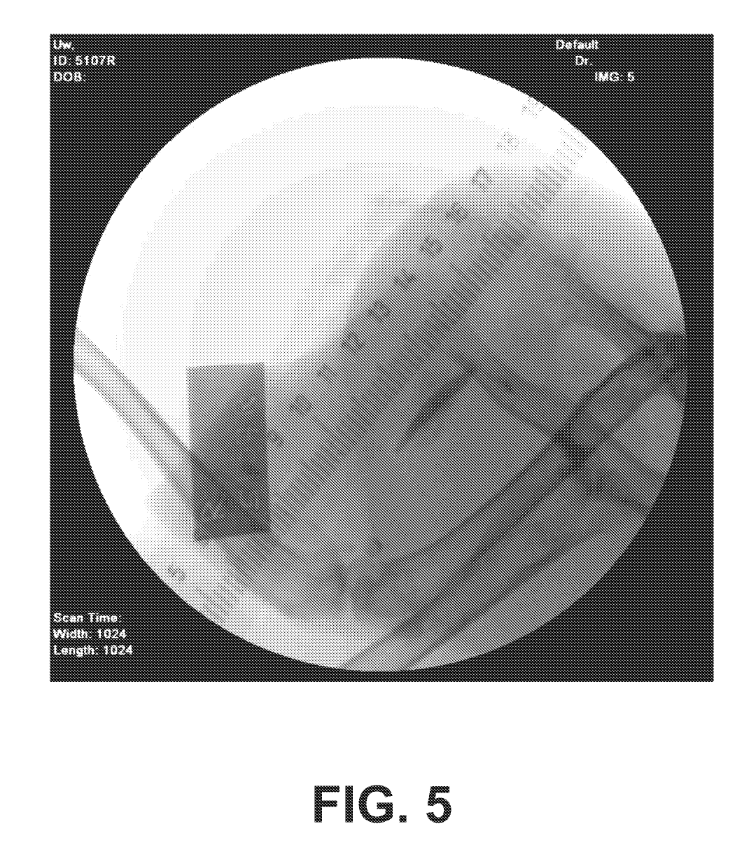

Post-injection images demonstrated a heterogenous, hypoechoic change on ultrasound. Fluoroscopic imaging (FIG. 5) showed an oblong radiopaque injection in both the paraspinous and right leg, suggesting local infiltration after injection.

The animal stayed afebrile throughout its entire life. Observation was performed at Day 7, with small palpable masses in both legs, and ultrasound showed a heterogeneous, partly hypoechoic mass.

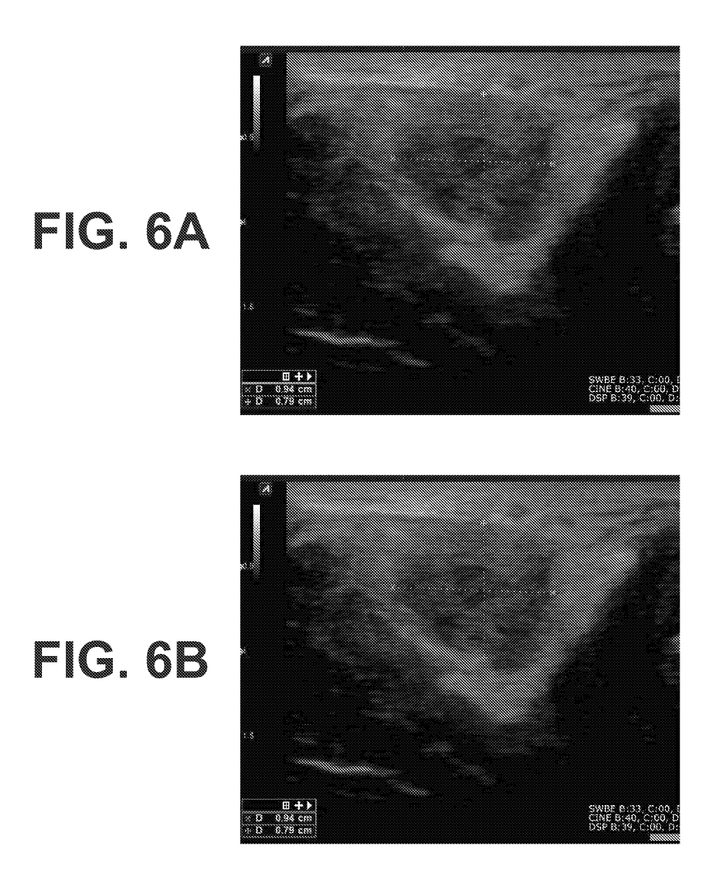

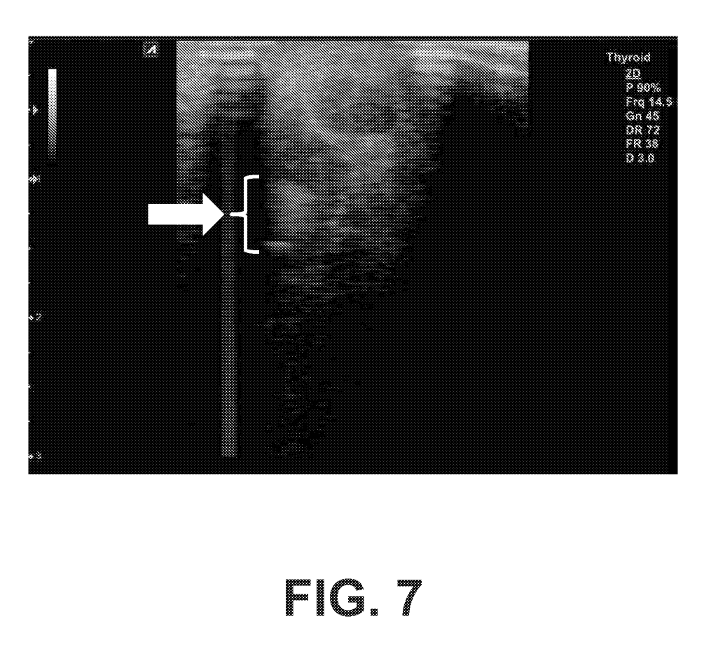

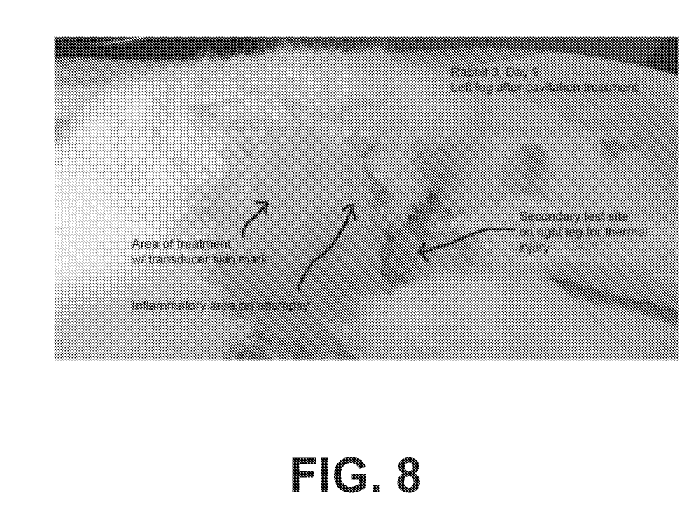

One lesion was identified on ultrasound (FIGS. 6A-6B) and treated for 5 minutes with a cavitation regime only (FIG. 7; 350 cycle pulses, 3% duty factor, 5.6 watts, calculated pressures +45 MPa, −15 MPa). A CW thermal regime was applied on an adjacent part of the leg after the rabbit was euthanized. Skin surface temperatures reached >70° C. within 10 seconds of CW, and a well-defined thermal injury was observed within 30 seconds. As shown in FIG. 8, no thermal injury was seen on the skin of the area treated with the cavitation regime.

### Example 4

### Co-Focal HIFU and Passive Cavitation Detector System

To passively detect cavitation during HIFU therapy, the foci of the HIFU and passive cavitation detection (“PCD”) transducers should co-register, whether the HIFU source is mobile or stationary. A fixture is fabricated to hold both transducers in fixed spatial relationship to one another. The fixture is used submersed in a water tank, with the animal suspended vertically with its head out of water, or on the bench top, using a thin conformal water sack to maintain coupling to the compound curvature of the thigh.

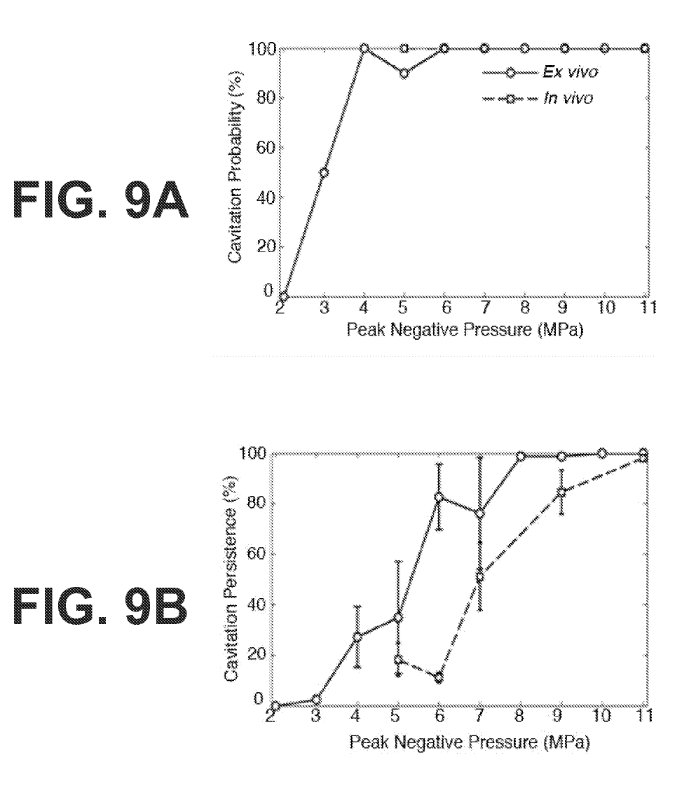

As a second form of passive (but qualitative) cavitation detection, diagnostic ultrasound is used to look for the hyperecho associated with cavitation. Cavitation-induced hyperecho is an accepted method for targeting and treatment monitoring in HIFU therapy. The PCD system is calibrated for quantifying inertial cavitation dose (which is technique-dependent, but can be quite precise) first in water before applying it to abscesses. FIGS. 9A-9B show data derived from PCD measurements in which a 1.1 MHz HIFU source was used to treat mouse pancreatic tumors in vivo or in vitro at various pressure amplitudes; cavitation probability was a measure of the frequency of occurrence of cavitation at previously unexposed sites; cavitation persistence was a measure of the probability that cavitation activity would occur repeatedly at the same site when insonated repeatedly.

### Example 5

### Determination of Microbial Viability in Pus Extracted from HIFU-Treated Abscesses

30 four-kg rabbits are inoculated substantially as described in Example 3. Bacterial viability in abscess pus sampled immediately before HIFU treatment will be compared to bacterial viability in abscess pus sampled on necropsy following treatment. Five animals will be assigned to each of three treatment arms: (i) ‘thermal only’ regime, using relatively low pressure amplitudes and high duty factors, (ii) ‘cavitation only’ regime, using very high pressure amplitudes at very low duty factors, and (iii) ‘thermal plus cavitation’ regime, using relatively high duty factors and high pressure amplitudes. The bactericidal impact of the treatments will be determined in relation to both the cavitation dose and the thermal dose individually (arms (i) and (ii)) or together (arm (iii)).

### Example 6

### Assessment of Abscess Treatment Efficacy in Survival Studies

Treatable abscesses are created in 15 four-kg rabbits substantially as described in Example 3. Based on the data obtained in Example 5, ten animals will be treated with HIFU and five will be sham-exposed as controls.

After treatment/sham-treatment, the animals will be recovered and returned to housing. Diagnostic ultrasound will be used to assess abscess regression/shrinkage during a one-week survival period. Systemic health will be monitored at 1, 2, 3, 5 and 7 days post treatment by measuring body temperature, white cell counts and endotoxin levels if other indicators suggest the need.

Seven days after treatment, the animals will be euthanized and necropsied. Pus samples are recovered from each animal and are subjected subsequently to viability assays and to histological evaluation. At necropsy, the size of the abscess will be directly measured (e.g., wall-to-wall diameter measurements across several transects), and samples of the abscess capsule and of adjacent tissues will be collected to evaluate whether the HIFU treatment had created significant collateral damage. A complete histomorphological evaluation of the tissue will be performed blindly by a board-certified pathologist.

Statistical tests will include two-sample comparisons of treated and sham controls, and will use (i) abscess size measurements collected by diagnostic ultrasound during the survival period, (ii) size measurements taken at necropsy, (iii) size measurements taken on histology, and (iv) bacterial viability (HA: viability with HIFU <viability with sham treatment).

### Further Examples

1. A method of treating an abscess associated with a subject, the method comprising:
applying therapeutic acoustic energy to the abscess,
wherein the therapeutic acoustic energy is sufficient to disrupt and/or destroy at least a portion of a pathogenic component of the abscess.

2. The method of example 1 wherein the abscess is associated with appendicitis, pancreatitis, cholecystectomy or gallbladder perforations, biliary leakage, gastrointestinal perforations, enteric fistulas, hernias and volvulus, diverticulitis, intussusceptions, post-operative infections or leakages, malignancies, ischemia and embolic disease, vasculitis, trauma, local radiation therapy or brachytherapy, a localized infection or abscess within a solid organ, a localized infection or abscess within a subcutaneous soft tissue, cystitis, pyelonephritis, urethritis, prostatitis, a genitourinary abscess, an infection after surgical revisions such as a diverting ileostomy, a neobladder, an infection associated with a nephrostomy stent/drain, an infection associated with a suprapubic drains, an infection associated with a stone, an infection associated with a malignancy, an infection associated with a trauma, an infection associated with a fistulas, an infection associated with a bladder/ureteric perforation, acute appendicitis, acute cholecystitis, mastitis, cellulitis, erysipelas, a thermal burn, a chemical burn, a breast malignancy, a skin malignancy, lymphoma, a mycobacterium, an allergic reaction, a lymphatic obstruction, a surgery, a biopsy, a piercing, a tattoo, a venous obstruction, an abscess within a rectus abdominis muscle, an abscess within a transverse abdominis muscle, an abscess within a psoas muscle, an abscess within a levator ani muscle, an abscess within a piriformis muscle, an abscess within an obturator muscle, an abscess within an adductor muscle, an abscess within a gluteal muscle, an abscess within a muscle of a shoulder girdle, an abscess within a rotator cuff, an abscess within a chest wall, an abscess within a pectus muscle, an abscess within a serratus muscle, an abscess within a sternocleidomastoid muscle, an abscess within a latissimus dorsi muscle, an abscess within a trapezius muscle, an abscess within a biceps muscle, an abscess within a triceps brachii muscle, an abscess within a deltoid muscle, parapneumonic effusion, empyema, chylothorax, pericarditis, or mediastinitis.

3. The method of example 1 or example 2 wherein the therapeutic acoustic energy is focused on only a portion of the abscess.

4. The method of any one of examples 1 to 3 wherein the therapeutic acoustic energy comprises a plurality of energy pulses.

5. The method of any one of examples 1 to 4 wherein the therapeutic acoustic energy is applied transcutaneously.

6. The method of any one of examples 1 to 5 wherein the therapeutic acoustic energy is focused within the abscess.

7. The method of any one of examples 1 to 6 wherein tissue surrounding the abscess is not damaged.

8. The method of any one of examples 1 to 7, further comprising obtaining an image of the abscess before applying the therapeutic acoustic energy to the abscess.

9. The method of example 8 wherein the image of the abscess is obtained by ultrasound, computed tomography or magnetic resonance imaging.

10. The method of example 8 wherein the image of the abscess is an ultrasound image obtained using an ultrasound transducer array configured to additionally generate the therapeutic acoustic energy.

11. The method of any one of examples 1 to 10 wherein the therapeutic acoustic energy comprises high intensity focused ultrasound energy.

12 The method of any one of examples 1 to 11 wherein the method does not include draining the abscess.

13. The method of example 11 or 12 wherein the therapeutic acoustic energy is applied for at least about 3 minutes.

14. The method of any one of examples 11 to 13 wherein the therapeutic acoustic energy has a peak pressure of about −20 MPa to about 1000 MPa.

15. The method of any one of examples 1 to 14 wherein the therapeutic acoustic energy disrupts and/or destroys at least a portion of the pathogenic component of the abscess by increasing a temperature of the pathogenic component.

16. The method of any one of examples 1 to 15 wherein the therapeutic acoustic energy disrupts and/or destroys at least a portion of the pathogenic component of the abscess by mechanically disrupting the pathogenic component.

17. The method of any one of examples 1 to 16 wherein the abscess comprises more than one abscess.

18. The method of any one of examples 1 to 17 wherein the abscess comprises a septated abscess.

19. The method of any one of examples 1 to 18 wherein the abscess comprises an infected fluid collection.

20. A method of inducing cavitation in an abscess, the method comprising:
applying high-intensity focused ultrasound energy focused within at least a portion of the abscess,
wherein a temperature associated with the abscess is not significantly increased during the step of applying the high-intensity focused ultrasound energy.

21. The method of example 20 wherein the high-intensity focused ultrasound energy is applied in an amount sufficient to disrupt and/or destroy at least a portion of a pathogenic component of the abscess.

22. The method of example 20 or 21 wherein the high-intensity focused ultrasound energy is applied for at least about 3 minutes.

23. The method of any one of examples 20 to 22 wherein the therapeutic acoustic energy disrupts and/or destroys at least a portion of the pathogenic component of the abscess by increasing a temperature of the pathogenic component.

24. The method of any one of examples 20 to 23 wherein the therapeutic acoustic energy disrupts and/or destroys at least a portion of the pathogenic component of the abscess by mechanically disrupting the pathogenic component.

25. The method of any one of examples 20 to 24 wherein the abscess comprises more than one abscess.

26. The method of any one of examples 20 to 25 wherein the abscess comprises a septated abscess.

27. The method of any one of examples 20 to 26 wherein the abscess comprises an infected fluid collection.

28. An acoustic abscess ablation system, comprising:
a signal generator configured to generate an ultrasound waveform;
an amplifier in operative communication with the signal generator for converting the ultrasound waveform into a high-intensity ultrasound waveform having an increased intensity to a signal transducer; and
a signal transducer having an adjustable focus for delivering the high-intensity ultrasound waveform to an abscess within a patient,
wherein the high-intensity ultrasound waveform is sufficient to disrupt and/or destroy at least a portion of a pathogenic component of the abscess.

29. The acoustic ablation system of example 28 wherein the system further comprises a second transducer for obtaining an ultrasound image of the abscess.

30. The acoustic ablation system of example 29 wherein the signal transducer and the second transducer are housed in a single transducer housing.

31. The acoustic ablation system of example 29 or example 30, further comprising a display for displaying the ultrasound image of the abscess.

32. The acoustic ablation system of example 29 wherein the second transducer is a diagnostic ultrasound transducer.

33. The acoustic ablation system of example 29 or example 32 wherein the signal transducer is in a first housing and the second transducer is in a second, separate housing.

34. The acoustic ablation system of example 29 or example 32 wherein the signal transducer and the second transducer are in a single housing.

35. The acoustic ablation system of any one of examples 28 to 34 wherein the therapeutic acoustic energy disrupts and/or destroys at least a portion of the pathogenic component of the abscess by increasing a temperature of the pathogenic component.

36. The acoustic ablation system of any one of examples 28 to 35 wherein the therapeutic acoustic energy disrupts and/or destroys at least a portion of the pathogenic component of the abscess by mechanically disrupting the pathogenic component.

37. The acoustic ablation system of any one of examples 28 to 36 wherein the abscess comprises more than one abscess.

38. The acoustic ablation system of any one of examples 28 to 37 wherein the abscess comprises a septated abscess.

39. The acoustic ablation system of any one of examples 28 to 38 wherein the abscess comprises an infected fluid collection.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the invention have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

1. A method of treating an abscess associated with a subject, the method comprising:
applying therapeutic acoustic energy to the abscess,
wherein the therapeutic acoustic energy is sufficient to disrupt and/or destroy at least a portion of a pathogenic component of the abscess.

2. The method of claim 1 wherein the abscess is associated with appendicitis, pancreatitis, cholecystectomy or gallbladder perforations, biliary leakage, gastrointestinal perforations, enteric fistulas, hernias and volvulus, diverticulitis, intussusceptions, post-operative infections or leakages, malignancies, ischemia and embolic disease, vasculitis, trauma, local radiation therapy or brachytherapy, a localized infection or abscess within a solid organ, a localized infection or abscess within a subcutaneous soft tissue, cystitis, pyelonephritis, urethritis, prostatitis, a genitourinary abscess, an infection after surgical revisions such as a diverting ileostomy, a neobladder, an infection associated with a nephrostomy stent/drain, an infection associated with a suprapubic drains, an infection associated with a stone, an infection associated with a malignancy, an infection associated with a trauma, an infection associated with a fistulas, an infection associated with a bladder/ureteric perforation, acute appendicitis, acute cholecystitis, mastitis, cellulitis, erysipelas, a thermal burn, a chemical burn, a breast malignancy, a skin malignancy, lymphoma, a mycobacterium, an allergic reaction, a lymphatic obstruction, a surgery, a biopsy, a piercing, a tattoo, a venous obstruction, an abscess within a rectus abdominis muscle, an abscess within a transverse abdominis muscle, an abscess within a psoas muscle, an abscess within a levator ani muscle, an abscess within a piriformis muscle, an abscess within an obturator muscle, an abscess within an adductor muscle, an abscess within a gluteal muscle, an abscess within a muscle of a shoulder girdle, an abscess within a rotator cuff, an abscess within a chest wall, an abscess within a pectus muscle, an abscess within a serratus muscle, an abscess within a sternocleidomastoid muscle, an abscess within a latissimus dorsi muscle, an abscess within a trapezius muscle, an abscess within a biceps muscle, an abscess within a triceps brachii muscle, an abscess within a deltoid muscle, parapneumonic effusion, empyema, chylothorax, pericarditis, or mediastinitis.

3. The method of claim 1 wherein the therapeutic acoustic energy is focused on only a portion of the abscess.

4. The method of claim 1 wherein the therapeutic acoustic energy comprises a plurality of energy pulses.

5. The method of claim 1 wherein the therapeutic acoustic energy is applied transcutaneously.

6. The method of claim 1 wherein the therapeutic acoustic energy is focused within the abscess.

7. The method of claim 1 wherein tissue surrounding the abscess is not damaged.

8. The method of claim 1, further comprising obtaining an image of the abscess before applying the therapeutic acoustic energy to the abscess.

9. The method of claim 8 wherein the image of the abscess is obtained by ultrasound, computed tomography or magnetic resonance imaging.

10. The method of claim 8 wherein the image of the abscess is an ultrasound image obtained using an ultrasound transducer array configured to additionally generate the therapeutic acoustic energy.

11. The method of claim 1 wherein the therapeutic acoustic energy comprises high intensity focused ultrasound energy.

12. The method of claim 1 wherein the method does not include draining the abscess.

13. The method of claim 11 wherein the therapeutic acoustic energy is applied for at least about 3 minutes.

14. The method of claim 11 wherein the therapeutic acoustic energy has a peak pressure of about −20 MPa to about 1000 MPa.

15. (canceled)

16. (canceled)

17. (canceled)

18. (canceled)

19. (canceled)

20. A method of inducing cavitation in an abscess, the method comprising:
applying high-intensity focused ultrasound energy focused within at least a portion of the abscess,
wherein a temperature associated with the abscess is not significantly increased during the step of applying the high-intensity focused ultrasound energy.

21. The method of claim 20 wherein the high-intensity focused ultrasound energy is applied in an amount sufficient to disrupt and/or destroy at least a portion of a pathogenic component of the abscess.

22. The method of claim 20 wherein the high-intensity focused ultrasound energy is applied for at least about 3 minutes.

23. (canceled)

24. (canceled)

25. (canceled)

26. (canceled)

27. (canceled)

28. An acoustic abscess ablation system, comprising:
a signal generator configured to generate an ultrasound waveform;
an amplifier in operative communication with the signal generator for converting the ultrasound waveform into a high-intensity ultrasound waveform having an increased intensity to a signal transducer; and
a signal transducer having an adjustable focus for delivering the high-intensity ultrasound waveform to an abscess within a patient,
wherein the high-intensity ultrasound waveform is sufficient to disrupt and/or destroy at least a portion of a pathogenic component of the abscess.

29. The acoustic ablation system of claim 28 wherein the system further comprises a second transducer for obtaining an ultrasound image of the abscess.

30. The acoustic ablation system of claim 29 wherein the signal transducer and the second transducer are housed in a single transducer housing.

31. The acoustic ablation system of claim 29, further comprising a display for displaying the ultrasound image of the abscess.

32. The acoustic ablation system of claim 29 wherein the second transducer is a diagnostic ultrasound transducer.

33. The acoustic ablation system of claim 29 wherein the signal transducer is in a first housing and the second transducer is in a second, separate housing.

34. The acoustic ablation system of claim 29 wherein the signal transducer and the second transducer are in a single housing.

35. (canceled)

36. (canceled)

37. (canceled)

38. (canceled)

39. (canceled)
